(19) United States
(12) Patent Application Publication
(10) Pub. No.: US 2024/0024087 A1
(43) Pub. Date: Jan. 25, 2024

(54) CONTACTLESS BREAST IMPLANT DELIVERY SYSTEM WITH FUNNEL AND METHOD OF PROVIDING AND USING THE SAME

(71) Applicant: Airtite Enterprises, LLC, Phoenix, AZ (US)

(72) Inventors: Amanda Swanson, Phoenix, AZ (US); Steven Sigalove, Paradise Valley, AZ (US)

(73) Assignee: Airtite Enterprises, LLC, Phoenix, AZ (US)

(21) Appl. No.: 17/869,762

(22) Filed: Jul. 20, 2022

(51) Int. Cl. A61F 2/00; A61F 2/12

(52) U.S. Cl. CPC A61F 2/0095; A61F 2/12; A61B 17/3468

(57) ABSTRACT

An implantation system for placing an implant into a patient during a surgical procedure wherein the implant is sterile is described. The implantation system can include: (a) a first container with a first side and a second side and configured to hold the implant inside the first container; and (b) a funnel with a first end and a second end. An inner surface of the first container and an inner surface of the funnel are sterile. The first end of the funnel is coupled to the second side of the first container such that the implant can move inside the funnel from the first container and the funnel is further configured such that the implant can be removed from the funnel at the second end of the funnel. The first container and the funnel are configured such that the sterility of the implant is maintained until the implant is removed the second end of the funnel. Other embodiments are disclosed.

### CROSS-REFERENCE TO RELATED APPLICATIONS

This application claims the benefit of U.S. Provisional Application No. 43/336,555 filed Jul. 24, 2021.

### FIELD OF THE INVENTION

This invention relates generally implantation system for facilitating insertion of an implant into a patient and relates more particularly to breast implantation systems and methods of providing and using the same.

### DESCRIPTION OF THE BACKGROUND

Breast augmentation surgery is one of the top five most commonly performed plastic surgery procedures in the world. In 2021, doctors performed approximately 365,000 breast augmentation procedures, which made it the second most popular plastic surgery behind liposuction.

In general breast augmentation surgery is safe and complications have become more infrequent because of improved surgical procedures and techniques. However, capsular contracture has become the number one complication/side effect following breast augmentation surgery in the past decade. The medical community and device manufacturers have struggled to define appropriate treatments and protocols to minimize this common issue. Literature shows up to 30% capsular contracture rate following primary breast augmentation.

In addition to capsular contracture, bacterial contamination of the implant and the surgery site at the time of surgery can lead to formation of a biofilm and capsular contracture along with infection. In many cases, the bacterial contamination is caused by human contact with the implant or contact between surgical instruments and the implant, exposure to open air, and/or contact with skin when performing breast augmentations. Doctors have developed detailed processes and methods (including white paper studies) to minimize potential infection but while they have decreased infections, bacterial contamination and its effects are still large problems for breast augmentation procedures.

Accordingly, a need or potential for benefit exists for an apparatus, system and method that decrease the potential for bacterial and other contaminations, decrease formation of biofilms and capsular contracture during breast augmentation surgeries.

### BRIEF DESCRIPTION OF THE DRAWINGS

To facilitate further description of the embodiments, the following drawings are provided in which:

FIG. 1 illustrates a cut away view of a breast implant system according to a first embodiment.

FIG. 2 illustrates a blown-up view of a breast implantation system, according to a second embodiment.

FIG. 3 illustrates a top view of part of the breast implantation, according to the second embodiment.

FIG. 4 illustrates a side view of part of the breast implantation system, according to the second embodiment.

FIG. 5 illustrates an isometric view of part of the breast implantation system, according to the second embodiment.

FIG. 6 illustrates a section view, along line B-B (FIG. 3) of part of the breast implantation system, according to the second embodiment.

FIG. 7 illustrates an isometric view of part of the breast implantation system with a funnel extended, according to a second embodiment.

FIG. 8 illustrates a flow chart for an embodiment of a method of producing an implantation system for an implant, according to an embodiment.

FIG. 9 illustrates a flow chart for an exemplary embodiment of activity of providing a frame of the method of FIG. 8, according to an embodiment.

FIG. 10 illustrates an isometric view of a dome and a frame before coupling of the dome and the frame, according to an embodiment.

FIG. 11 illustrates an isometric view of a dome and a frame after coupling of the dome and the frame, according to an embodiment.

FIG. 12 illustrate an isomeric view of a funnel during the manufacturing process, according to an embodiment.

FIG. 13 illustrates another isometric view of a funnel later in the manufacturing process, according to an embodiment.

FIG. 14 illustrates an isometric view of a funnel and a ring before coupling, according to an embodiment.

FIG. 15 illustrates an isometric view of a funnel and a ring after coupling, according to an embodiment.

FIG. 16 illustrates an isometric view of the lubricating of a funnel, according to an embodiment.

FIG. 17 illustrates an isometric view of the lubricating of a frame and a dome, according to an embodiment.

FIG. 18 illustrate an isometric view of first container and an implant before placing of the implant into a dome, according to an embodiment.

FIG. 19 illustrates a cut-away view of a first container and an implant after the coupling of a ring to a frame, according to an embodiment.

FIG. 20 illustrates an isometric view of a frame and an inner cover before coupling, according to an embodiment.

FIG. 21 illustrates a cut-away view of a frame and an inner cover after coupling, according to an embodiment.

FIG. 22 illustrates a flow chart for an exemplary embodiment of activity of providing an outer container of the method of FIG. 8 according to an embodiment.

FIG. 23 illustrates an isometric view of a breast implantation system before outer a cover is coupled to an outer tray, according to an embodiment.

FIG. 24 illustrates a cut-away view of a breast implantation system, according to an embodiment.

FIG. 25 illustrates a flow chart for an embodiment of a method of placing a sterile implant in a patient at a point of insertion.

FIG. 26 illustrates an isometric view of the opening of an outer container, according to an embodiment.

FIG. 27 illustrates a partial view of a frame and an outer tray with one or more finger pickers, according to an embodiment.

FIG. 28 illustrates an isometric view of the opening of a first container, according to an embodiment.

FIG. 29 illustrates an isometric view of the injecting of liquid using a syringe into a dome using one of the ports in a frame, according to an embodiment.

FIG. 30 illustrates a cut-away close up view of the injecting of liquid using a syringe into a dome using one of ports in a frame, according to an embodiment.

FIG. 31 illustrates an isometric view of a first container during a rotation process, according to an embodiment.

FIG. 32 illustrates an isometric view of a first container after rotation, according to an embodiment.

FIG. 33 illustrates a cut-away view of a first container before descension of an implant, according to an embodiment.

FIG. 34 illustrates a cut-away view of a first container during descension of an implant, according to an embodiment.

FIG. 35 illustrates a cut-away view of a first container after descension of an implant, according to an embodiment.

FIG. 36 illustrates an isometric view of a first container before detachment of a funnel, according to an embodiment.

FIG. 37 illustrates an isometric view of a first container during detachment of a funnel, according to an embodiment.

FIG. 38 illustrates an isometric view of a funnel after detachment of a funnel, according to an embodiment.

For simplicity and clarity of illustration, the drawing figures illustrate the general manner of construction, and descriptions and details of well-known features and techniques may be omitted to avoid unnecessarily obscuring the invention. Additionally, elements in the drawing figures are not necessarily drawn to scale. For example, the dimensions of some of the elements in the figures may be exaggerated relative to other elements to help improve understanding of embodiments of the present invention. The same reference numerals in different figures denotes the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the invention described herein are, for example, capable of operation in other orientations than those illustrated or otherwise described herein.

The terms “couple,” “coupled,” “couples,” “coupling,” and the like should be broadly understood and refer to connecting two or more elements or signals, electrically, mechanically and/or otherwise. Coupling may be for any length of time, e.g., permanent or semi-permanent or only for an instant.

“Mechanical coupling” and the like should be broadly understood and include mechanical coupling of all types.

The absence of the word “removably,” “removable,” and the like near the word “coupled,” and the like does not mean that the coupling, etc. in question is or is not removable.

### DETAILED DESCRIPTION OF EXAMPLES OF EMBODIMENTS

In a number of embodiments, an implantation system for placing an implant into a patient during a surgical procedure wherein the implant is sterile is described. The implantation system can include: (a) a first container with a first side and a second side and be configured to hold the implant inside the first container; and (b) a funnel with a first end and a second end. An inner surface of the first container and an inner surface of the funnel are sterile. The first end of the funnel is coupled to the second side of the first container such that the implant can move inside the funnel from the first container and the funnel is further configured such that the implant can be removed from the funnel at the second end of the funnel. The first container and the funnel are configured such that the sterility of the implant is maintained until the implant is removed the second end of the funnel.

In other embodiments, a method of producing an implantation system for an implant is described The method includes: (a) sterilizing the implant; (b) providing a first container with a first side and a second side; (c) providing a funnel with a first end and a second end where the funnel is further configured such that the implant can be removed from the funnel at the second end of the funnel; (d) sterilizing at least an inner surface of the first container and an inner surface of the funnel; (e) placing the implant into the first container; and (f) coupling the first container to the funnel such that the first end of the funnel is coupled to the second side of the first container such that the implant can move inside the funnel from the first container and that the sterility of the implant is maintained until the implant is removed the second end of the funnel.

In yet another embodiment, a method of placing a sterile implant in a patient at a point of insertion is described. The method includes: (a) unsealing a first container to expose a sterile funnel wherein sterile first container includes (1) a sterile funnel with a first end and a second end; and (2) a sterile first dome with a first side and a second side with the implant inside the first container; (b) moving the implant from the sterile first dome to the sterile funnel without any human contact with the implant; and (c) using the funnel to move the implant into the patient without any human contact to the implant other than contact with the patient at the point of insertion.

While the systems and methods described herein are described in relation to human breast implantation, the systems and methods can be use in other species and other types of implants. For example, these systems and methods can be used for other implants such as gluteal or calf implants or used in other specialties such as veterinary or orthopedic surgery.

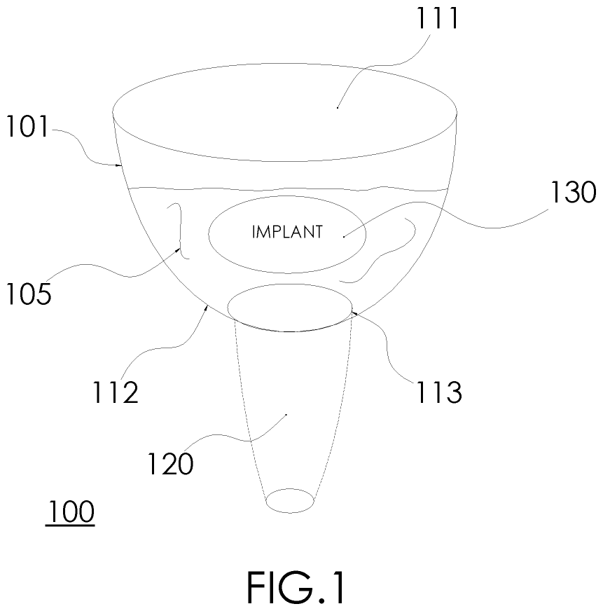

Turning to the drawings, FIG. 1 illustrates a cut away view of the breast implant system 100 according to a first embodiment. Breast implant system 100 is merely exemplary and is not limited to the embodiments presented herein. Breast implant system 100 can be employed in many different embodiments or examples not specifically depicted or described herein.

The purpose of breast implant system 100 and other embodiments herein is to provide a sterile packaging for breast implants that allows for contactless insert of the breast implant into the patient. Breast implant system 100 also can include an attached funnel for moving the implant easily and safely into the breast pocket of a patient (not shown). In many examples, the patient is a human. In other examples, the patient can be an animal.

As shown in FIG. 1, breast implant system 100 can include a container 101. Container 101 can house a silicone breast implant 130 of various sizes and profiles. In some examples, container 101 can comprises plastic or similar materials. In various embodiments, container 101 comprise a pliable plastic, with a sealed sterile interior and peel back top 111. In some examples, top 111 can be a peel back thick paper, as there will be an option to open the container from the top, or insert a needle to add optional antibiotic, betadine, or any other additional medication selected by the surgeon.

In the same or different examples, bottom 112 of container 110 can have an opening 113 attached to a funnel 120. In some embodiments, opening 113 can be round. In other examples, opening 113 can have other shapes that correspond to the shape of the top of funnel 120. In many examples, funnel 120 is plastic. In other examples, funnel 120 can be made of similar or different materials. The funnel can be accordion-shaped and folded to the extent of being flat against the bottom of the container. The funnel will then be sealed closed with a removal sticker seal, that when peeled, the funnel to extend and engage. In some embodiments, the funnel can be retractable while not being detachable.

In many examples, opening 113 can be sealed by a peel off sticker. Once the peel off sticker is engaged over opening 113, funnel 120 can be attached. Funnel 120 can receive the implant and contents of container 101. In some examples, the mechanism of moving implant 130 to funnel 120 can be gravity. In other examples, it can be a combination of gravity and pressure from something some squeezing container 110 in such a fashion, that the implant is forced into funnel 120. In other examples, other mechanisms (e.g., mechanical mechanism) can be used to move implant 130 into funnel 120. Additionally, in some embodiments, a lubricant (for example, LubriLAST™ lubricant) can be used to coat the inside of the funnel. In many examples, the tip 121 of funnel 120 can be sized to match the size of implant 130 at its smallest point, as to make passing through funnel 120 easier. In other examples, the size ratio of funnel 120 to implant 130 can be different. For example, funnel 120 at its smallest point can be 10% or 20% larger than implant 130. Next, funnel 120 can then be placed into the breast pocket and squeezed. This will allow the implant to pass from the packaging/container into the body using a no human touch technique (i.e., contactless). That is, the implant slides from container 101 into funnel 120 and then into the chest of the patient without any human hands touching implant 130. In many embodiments, the breast implant will have saline in its container to keep the implant moist. The container may also use a lubricant or solution that may be an antiseptic as well as possess other qualities that will help the implant move easily through the funnel.

This embodiment can allow for sterile packaging of a breast implant as well as insertion into the patient's body without any human contact. The usage of this device can provide a significant benefit to the safety and health of the patient receiving the implant. Using a “no touch technique” has been shown to greatly reduce the risk of infection. Benefit to insertion through the funnel also allows to pass a large size implant through a small incision, reducing scarring and incision size. The ability to move the breast implant from a sterile packaging, directly into the breast pocket without exposure to air, or any physical contact is different than other methods of implant insertion commonly used. Additionally, methods using the device described here can cut down on an additional line-item cost in surgery, which will result in cost savings. That is, the currently used funnel is costly to the hospital or surgery center and is an added “line item” to the surgery bill. This method of delivery will also save time in the operating room, as the funnel is already sized to match the implant and requires no additional prep time to trim.

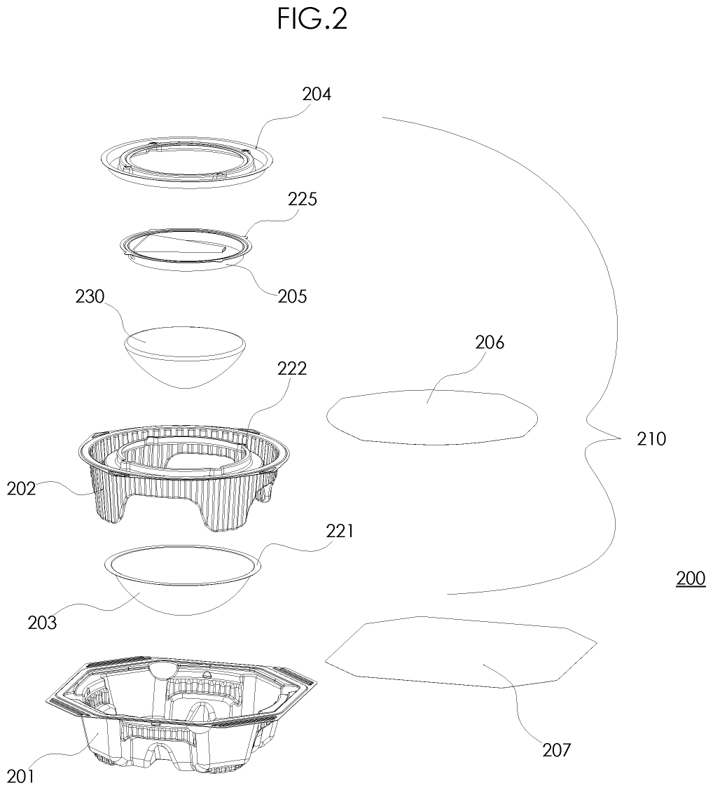

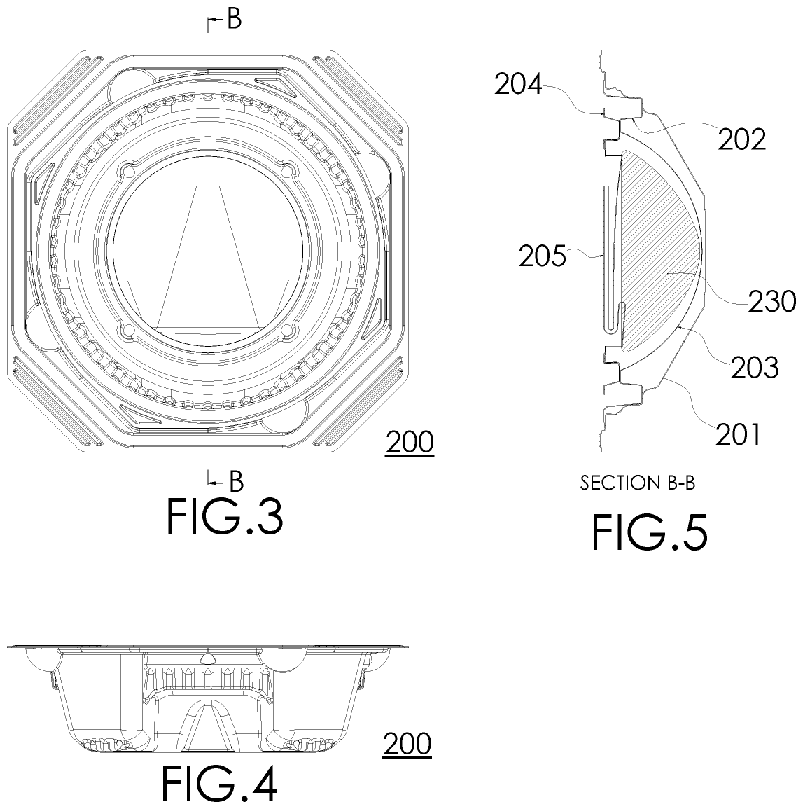

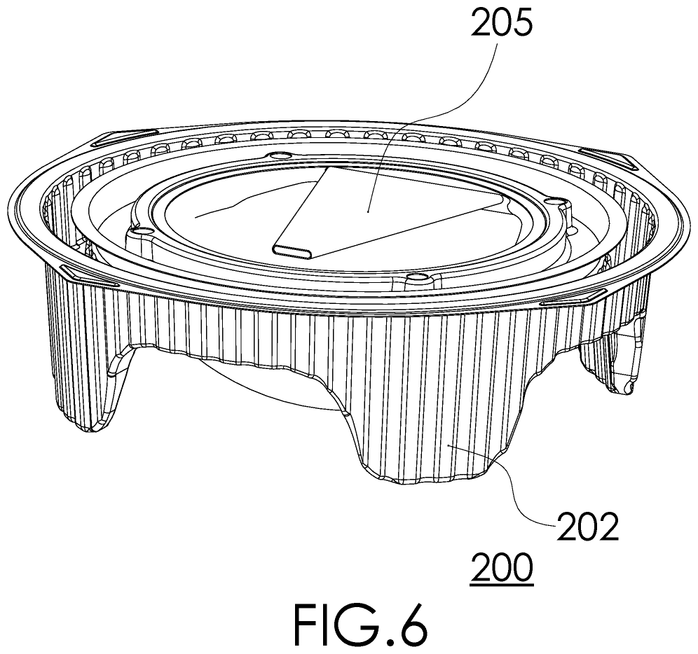

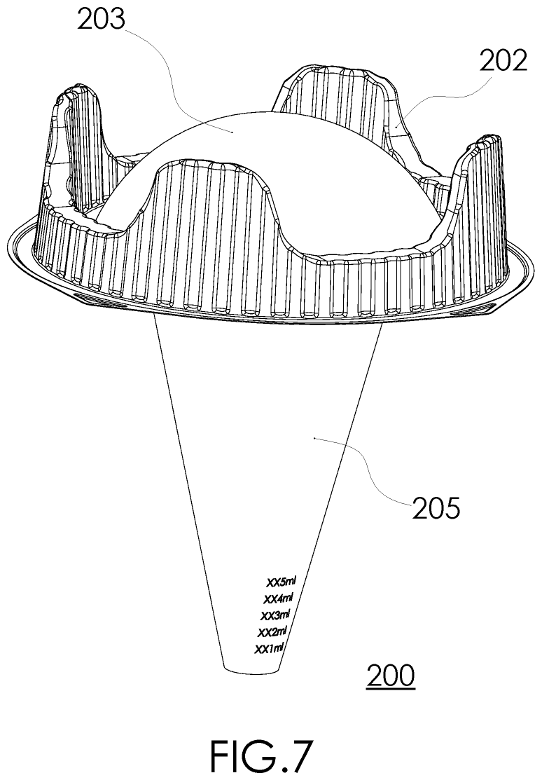

Turning to another example, FIG. 2 illustrates a blown-up view of a breast implantation system 200, according to a second embodiment. FIG. 3 illustrates a top view of breast implantation system (without inner cover 206 and outer cover 207), according to the second embodiment. FIG. 4 illustrates a side view of breast implantation system 200 (without inner cover 206 and outer cover 207), according to the second embodiment. FIG. 5 illustrates an isometric view of breast implantation system 200 (without inner cover 206 and outer cover 207), according to the second embodiment. FIG. 6 illustrates a section view, along line B-B (FIG. 3) (without outer tray 201, inner cover 206 and outer cover 207), according to the second embodiment. FIG. 7 illustrates an isometric view of breast implantation system 200 with funnel 205 extended (without outer tray 201, inner cover 206 and outer cover 207), according to a second embodiment. Breast implantation system 200 is merely exemplary and is not limited to the embodiments presented herein. Breast implantation system 200 can be employed in many different embodiments or examples not specifically depicted or described herein.

Referring to FIG. 2, breast implantation system 200 can include: (a) an outer tray 201; (b) a frame 202; (c) a dome 203; (d) a ring 204; (e) a funnel 205; (f) an inner cover 206; (g) an outer cover 207; and (h) an implant 230. In some examples, implant 230 is part of breast implantation system 200. In other examples, implant 230 is not part of breast implantation system 200 and is separate. In many embodiments, implant 230 is similar or the same as implant 130. In some examples, a first container 210 can include: frame 202, dome 203, ring 204, funnel 205 and/or inner cover 206.

In one example, breast implantation system 200 is 13.5 inches square when measured from the ends of frame 202 and 3.60 inches high. In other examples, breast implantation system 200 has one or more different dimensions.

In some embodiments, dome 203 can be made out of a plastic material. Dome 203 can be a clearly or semi-clear plastic to allow the surgeon and medical staff to see implant 230 in dome 203. For example, dome 203 can be a thermoplastics polyurethane. In the example shown in FIGS. 2-6, dome 203 is a half circular dome with a lip 221. In other examples, dome 203 can have other shapes such as a cylindrical shape and/or include other mechanism to mate to frame 202, funnel 205 and/or ring 204.

Frame 202 can be act as the main body of breast implantation system 200. As will be described herein, frame 202 can include one or more parts to allow dome 203, ring 204, funnel 205, inner cover 206, outer cover 207 and/or outer tray 201 to mate, couple, and/or otherwise connect to or abut frame 202 in a secure manner. For example, frame 202 can include a center opening with an inner ring 222 such that dome 203 can be inside of the opening. In many examples, frame 202 can be made out of a plastic material. In various embodiments, fame 202 can be made from a polyethylene terephthalate glycol material (PETG).

In some examples, breast implantation system 200 is assembled by placing dome 203 inside of frame 202. Dome 203 can have a lip 221 that is slightly larger than inner ring 222 of frame 202 such that the lip 221 rests on inner ring 222 when assembled, as shown in FIG. 6. In further examples, dome 203 can be surface welded to frame 202. In other examples, frame 202 and dome 203 can be one piece. In further embodiments other mechanism can be used to mate frame 202 to dome 203.

Implant 230 can be placed inside of dome 203 along with one or more liquids to preserve implant 230 and keep it sterile. In other embodiments, implant 230 can be placed inside of dome 203 without any liquids.

In various embodiments, funnel 205 can be located on top of dome 203 and frame 202. Ring 204 can be used to lock in place funnel 205 on top of frame 202 and/or dome 20. In some examples, funnel 205 can have an edge 223 that sits on top of inner ring 222. Ring 204 and frame 202 can have matching structures such that ring 204 can be placed around inner ring 222 to securely attach dome 203, frame 202, funnel and ring 204 together. In some examples, ring 204 can be a snap ring and ring can be placed inner ring 222 to create a secure pressure fit. In other examples, an adhesive or a surface weld may be used to couple ring 204 to frame 202 in addition to or instead of a pressure fit.

In many examples, ring 204 can be made of the same material as frame 202. In many examples, ring 204 can be made out of a plastic material. In various embodiments, ring 204 can be made from PETG.

In some examples, funnel 205 can be similar or the same as funnel 120. In many examples, funnel 205 is plastic. For example, funnel 205 can be made from TPU. In other examples, funnel 205 can be made of similar or different materials. Funnel 205 can be accordion-shaped and folded to the extent of being flat before being extended. In some examples, funnel 205 can be removalable from frame 202. In one example, funnel 205 can have perforations that allow a surgeon or nurse to pinch a portion of funnel 205 and rotate from 202 to tear funnel 202 from frame 202 while implant 230 is located in funnel 205. In other examples, funnel 205 remains couple to frame 202 during placement of the implant 202 into the patient.

Inner cover 206 can be secured to frame 202 to seal funnel 205 and implant 230 inside of frame 202 and dome 203. In some examples, inner cover 206 can be a removal sticker seal. In some examples, inner cover 206 can be made from polyethylene fibers. In one example, inner cover can be made from a Tyvek® material, which is made by DuPont de Nemours, Inc.

Outer tray is located around dome 203, frame 202 and inner cover 206 to completely enclose dome 203, frame 202 and inner cover 206. Outer cover 207 can be similar to inner cover 206. Outer cover 207 cane be securely attached to outer tray 207 to complete enclose dome 203, frame 202 and inner cover 206. enclose dome 203, frame 202 and inner cover 206. Outer cover 207 can be attached to outer tray 207 such that outer cover 207 can be removed from outer tray 201. In some embodiments, outer cover 207 can include an edge or other portion that can be gripped by a person to peel outer 207 from outer tray 207.

In many examples, outer tray 207 can be made of the same material as frame 202. In many examples, outer tray 207 can be made out of a plastic material. In various embodiments, outer tray 207 can be made from PETG. In some examples, inner cover 206 can be made from polyethylene fibers. In one example, inner cover can be made from a Tyvek® material, which is made by DuPont de Nemours, Inc.

One of the advantages of the breast implantation system described herein is that the breast implantation system can be manufactured or assembled in a clean, sterile environment. This breast implantation system allows for a closed system, which minimizes bacterial presence in the frame and dome, and on the implant. As will be described below, this breast implantation system allows for a truly “no touch” technique where no person or surgical or medical tool touches the implant before insertion into the patient. Thus, the breast implantation systems and methods described herein will decrease bacterial (and other) infections and decreased biofilm along with capsular contracture. In addition to the health and safety benefits of the breast implantation systems and methods described herein, this system and method also has the benefit of decreasing costs related to breast implants to manufacturers. It is normal practice in the industry for the manufacturer of the implants to warrant the implants against capsular contracture. Because the system and methods described herein will decrease capsular contracture, warranty replacement and other costs incurred by the manufacturer or distributor can be also greatly decreased.

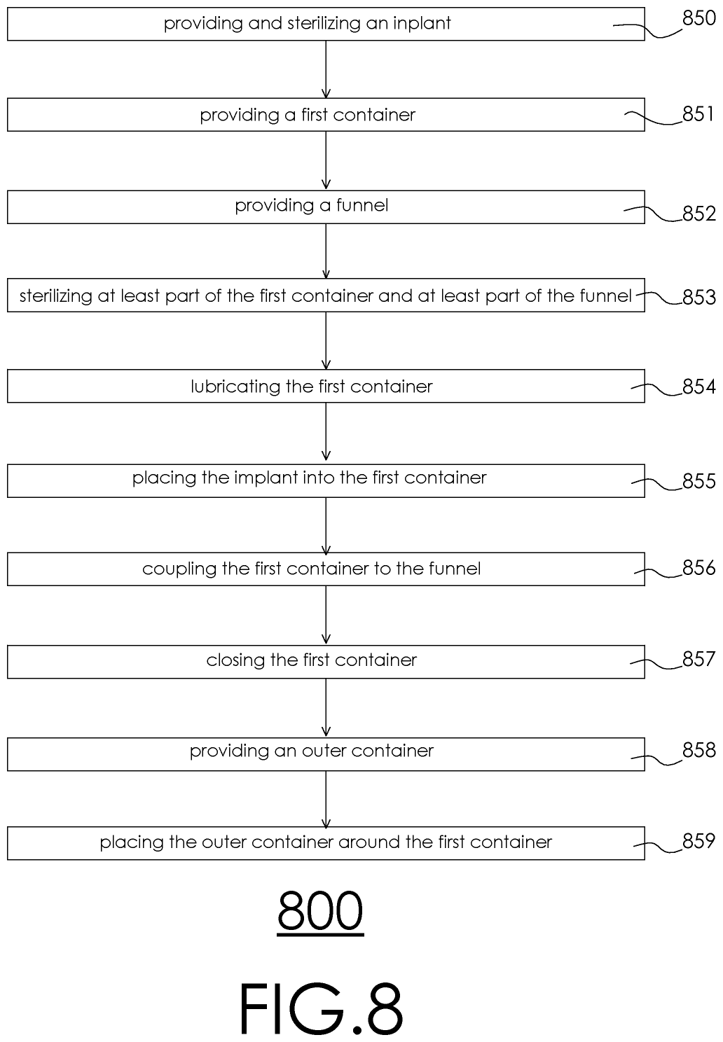

FIG. 8 illustrates a flow chart for an embodiment of a method 800 of producing an implantation system for an implant. Method 800 is merely exemplary and is not limited to the embodiments presented herein. Method 800 can be employed in many different embodiments or examples not specifically depicted or described herein. In some embodiments, the activities, the procedures, and/or the processes of method 800 can be performed in the order presented. In other embodiments, the activities, the procedures, and/or the processes of method 800 can be performed in any other suitable order. In still other embodiments, one or more of the activities, the procedures, and/or the processes in method 800 can be combined or skipped.

Referring to FIG. 8, method 1100 includes an activity 1110 of providing and sterilizing the implant. In some examples, the implant can be similar or identical to implants 130 of FIG. 1 or implant 230 of FIG. 2. In various embodiments, the implant can be manufactured or produced by methods well known in the art. In many examples, the implant is a breast implant. In other examples, the implant can be other types of implants such as a calf implant. The implant can be sterilized using method or systems that are well known in the art. For example, the implant can be sterilized using a chemical process to kill and/or remove all bacteria and other microbes. Additionally, all or some of the processes in method 800 can be performed in a clean room or sterile facility to maintain the sterility of the implant and/or other parts of the implantation system.

Method 800 in FIG. 8 continues with an activity 851 of providing a first container. As an example, the first container can be similar or identical to 101 of FIG. 1 or container 210 of FIG. 2. In some examples, providing the first container can involve either manufacturing or producing the first container or receiving the first container where such container was manufactured or produced at another facility or by another company.

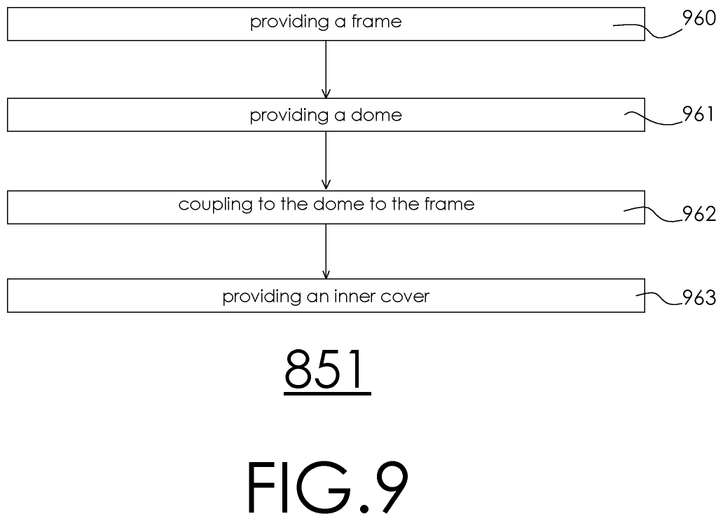

FIG. 9 illustrates a flow chart for an exemplary embodiment of activity 851 of method 800, according to an embodiment. Referring to FIG. 9, activity 820 includes a procedure 960 of providing a frame. As an example, the frame can be similar or identical to frame 202 of FIG. 2.

Subsequently, activity 851 of FIG. 9 includes a procedure 961 of providing a dome. As an example, the dome can be similar or identical to dome 105 or 203 of FIGS. 1 and 2, respectively.

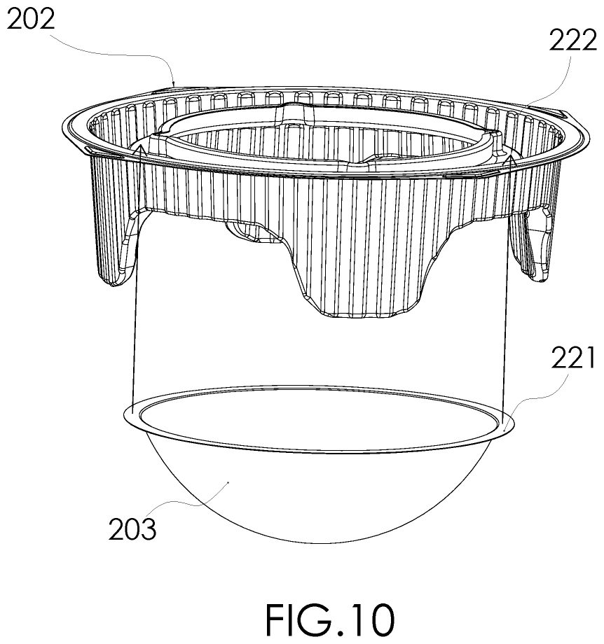

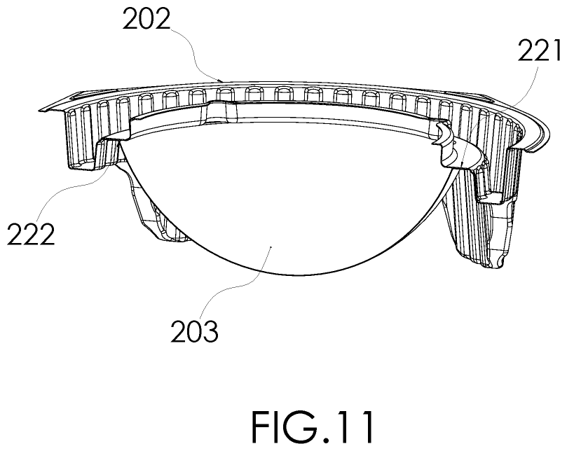

Next, activity 851 of FIG. 9 includes a procedure 962 of coupling the dome to the frame. FIG. 10 illustrates an isometric view of dome 203 and frame 202 before coupling of dome 203 and frame 202, according to an embodiment. FIG. 11 illustrates an isometric view of dome 203 and frame 202 after coupling of dome 203 and frame 202, according to an embodiment. As shown in FIGS. 10 and 11, frame 202 can be coupled to dome 203. In some examples, frame and dome can be coupled by surface welding them together. In one embodiment, lip 221 can be surface welded to inner ring 222.

Next, activity 851 of FIG. 9 includes a procedure 963 of providing an inner cover. In some examples, the inner cover can be similar or identical to inner cover 206 of FIG. 2.

Subsequently, method 800 of FIG. 8 includes an activity 852 of providing a funnel. As an example, the funnel can be similar or identical to funnel 120 or 205 of FIGS. 1 and 2, respectively.

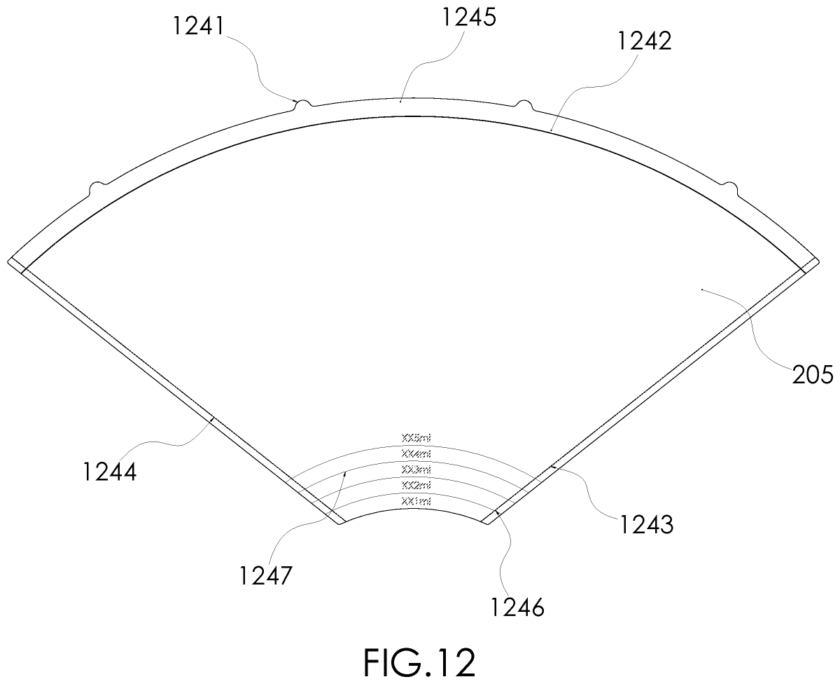

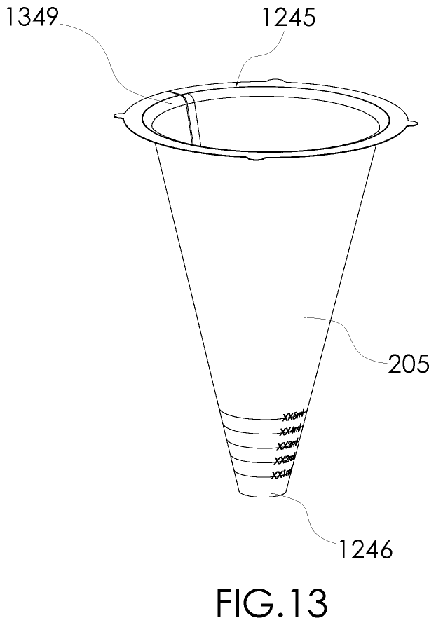

FIG. 12 illustrate an isomeric view of funnel 205 during the manufacturing process, according to an embodiment. FIG. 13 illustrates another isometric view of funnel 205 later in the manufacturing process, according to an embodiment. As shown in FIGS. 12 and 13, funnel 205 can a first side 1245 and a second side 1246. On first side 1245, funnel can include one or more flaps 1241 to facilitate liquid (e.g., Betadine) in be later inserted in the implantation system. Funnel 205 can also include a tear perforation to allow funnel 205 to be disconnected from frame 202 during the implantation surgery. In some examples, funnel 205 can also include graduation nomenclature marks 1247 near side 1246 to allow the surgeon or other medical professional to cut the funnel to the proper size. In some examples, marks 1247 can be laser etched.

In some embodiments, edge 1243 and be coupled to edge 1244 to create funnel 205. For example, edge 1243 can be seam welded to edge 1244. As shown in FIG. 13, the diameter of funnel 205 at side 1245 can be larger than the diameter of funnel 205 at edge 1246. In the same or different embodiments, an edge portion 1349 can be turned to allow funnel 205 to be coupled ring 204.

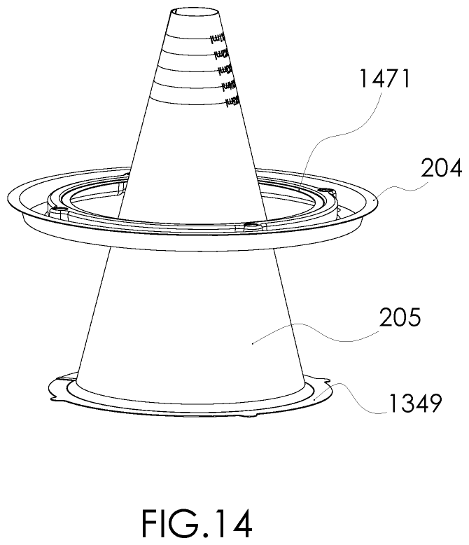

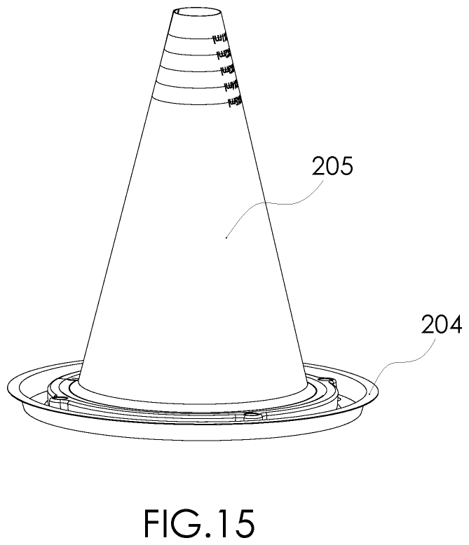

The next process in providing the funnel is coupling the funnel to a ring. FIG. 14 illustrates an isometric view of funnel 205 and ring 204 before coupling, according to an embodiment. FIG. 15 illustrates an isometric view of funnel 205 and ring 204 after coupling, according to an embodiment. In one example, edge portion 1349 of funnel 205 is coupled to edge portion 1471 of ring 204. In some embodiments, edge portion 1349 and edge portion 1471 are surface welded together. In another embodiment, edge portion 1349 and edge portion 1471 are glued together or coupled using another adhesive mechanism.

Next, method 800 of FIG. 8 includes an activity 853 of sterilizing at least part of the first container and at least part of the funnel. In some examples, an inner surface of the funnel 205 and an inner surface of frame 202 and an inner surface of dome 203 are sterilized. In other examples, all of funnel 205, frame 202 and dome 203 are sterilized. By sterilizing all surfaces that may touch implant 230, the sterility of the implant can be maintained. Also, by sterilizing all of funnel 205, frame 202 and dome 203 (and keeping them sterilized as will be described herein), the breast implantation system can be easily used in the sterile environment of the operating room, which will decrease the possibility of any bacterial or other infections. In some examples, one or more surfaces of inner cover 206 can also be sterilized during this activity.

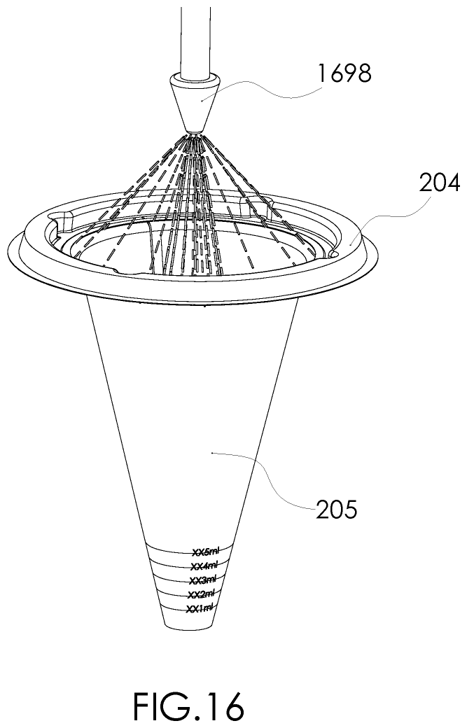

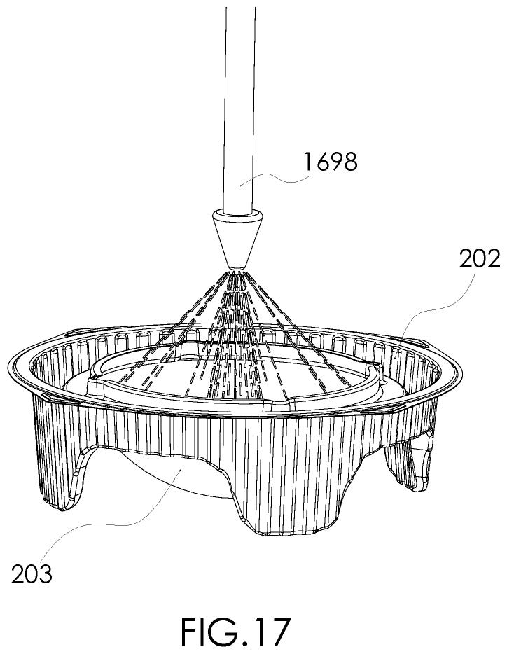

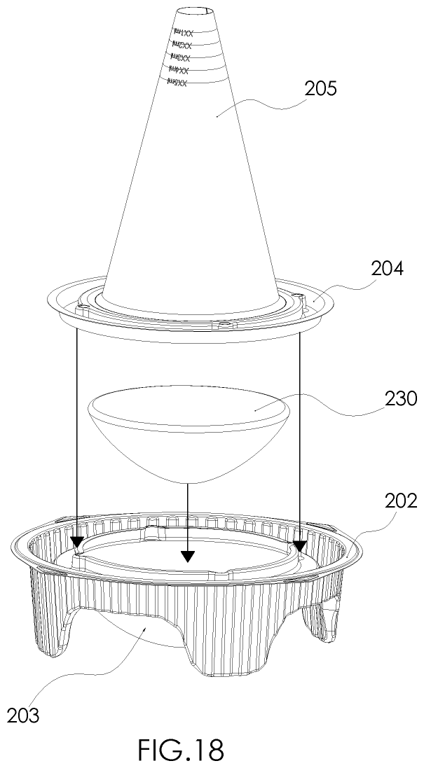

Next, method 800 of FIG. 8 includes an activity 854 of lubricating the first container and funnel. FIG. 16 illustrates an isometric view of the lubricating of funnel 205, according to an embodiment. FIG. 17 illustrates an isometric view of the lubricating of frame 202 and dome 203, according to an embodiment. In some examples, funnel 205, frame 202 and dome 203 can be lubricated by spraying a liquid into them using a spray nozzle 1698. In other embodiments, other mechanisms can be used to lubricate funnel 205, frame 202 and dome 203. As part of this activity, in some examples, the liquid lubricant can fully coat the inner surfaces of funnel 205, frame 202, and dome 203 which may contact or otherwise touch the implant. In one embodiment, the liquid can be a Lubrilast™ biocompatible lubricant. In other examples, other lubricating liquids can be used. Method 800 in FIG. 8 continues with an activity 855 of placing the implant into the first container. FIG. 18 illustrate an isometric view of frame 202, dome 203, ring 204, funnel 205, and implant 230 before placing of implant 230 into dome 203, according to an embodiment.

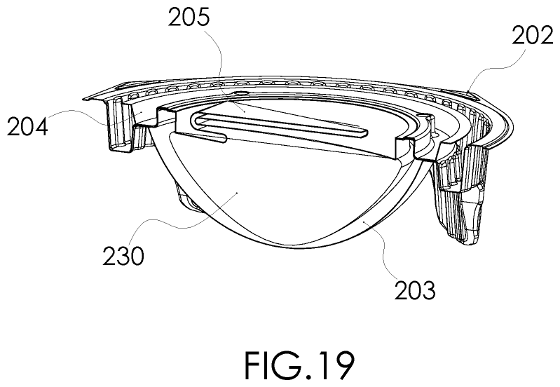

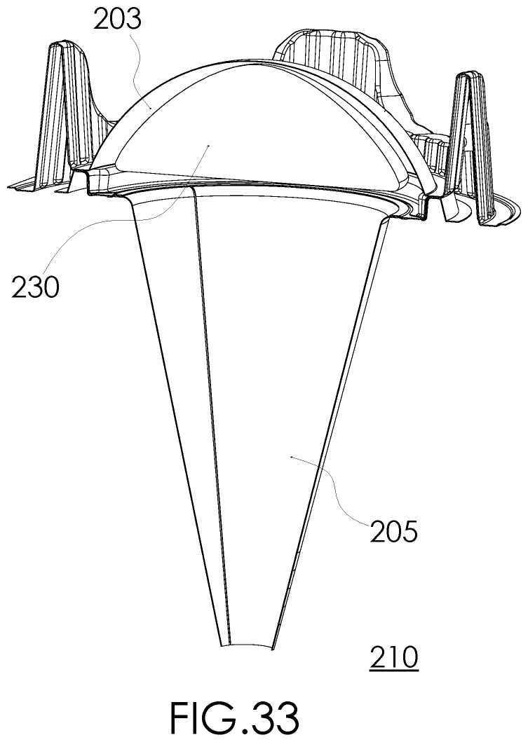

Subsequently, method 800 of FIG. 8 includes an activity 856 of coupling the first container to the funnel. FIG. 19 illustrates a cut-away view of frame 202, dome 203, ring 204, funnel 205, and implant 230 after the coupling of ring 204 to frame 202, according to an embodiment. In some embodiments, frame is couple to funnel 205 such that side 1245 (FIG. 13) of funnel 205 is coupled to a side of frame 202 such that implant 230 can move inside funnel 205 from dome 203 (via frame 202) (See FIGS. 33-35) and that the sterility of implant 230 is maintained until implant 230 is removed from end 1246 (FIG. 13) of funnel 205.

In some examples, an edge of ring 204 can be surface welded to an edge of frame 202. In other examples, ring 204 and frame 202 can be configured to use a snap ring to couple together. In this example, ring and frame can have a three-hundred-and-sixty-degree snap lock that securely couples ring 204 to frame 202. Additionally, as part of this activity, funnel 205 can be folded or otherwise compressed to allow sealing in the next activity and easier and sterile shipping and storage.

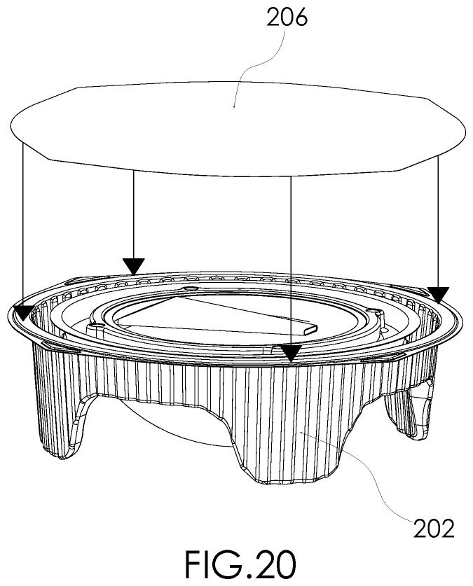

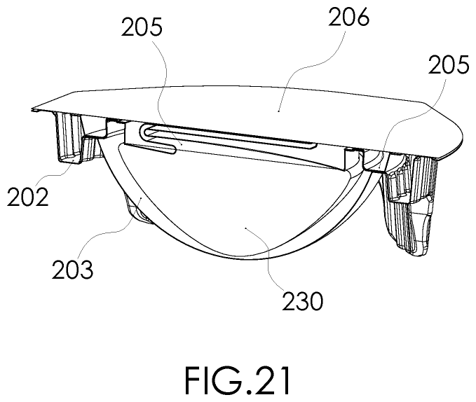

Subsequently, method 800 of FIG. 8 includes an activity 857 of closing the first container. In some examples, the first container is closed by removably coupling frame 202 to the inner cover 206 such that sterility of inner surfaces of funnel 205, dome 203 and frame 202 are maintained until inner cover 206 is removed from frame 202. FIG. 20 illustrates an isometric view of frame 202 and inner cover 206 before coupling, according to an embodiment. FIG. 21 illustrates a cut-away view of frame 202 and inner cover 206 after coupling, according to an embodiment. In some examples, inner cover 206 can be coupled to an edge (e.g., using a standard ⅜-inch flange) of frame 202. In one example, inner cover 206 can be surface welded to frame 202. In other examples, an adhesive can be used to coupled inner cover 206 to frame 202. When inner cover 206 is coupled to frame 202, a closed system is created and a sterile environment around implant 230 can be maintained until inner cover 206 is removed in the operating room. Keeping the system closed from the end of the manufacturing process until opened in the operating room will decrease the possibility of bacteria or other foreign materials getting on implant 230 before implantation.

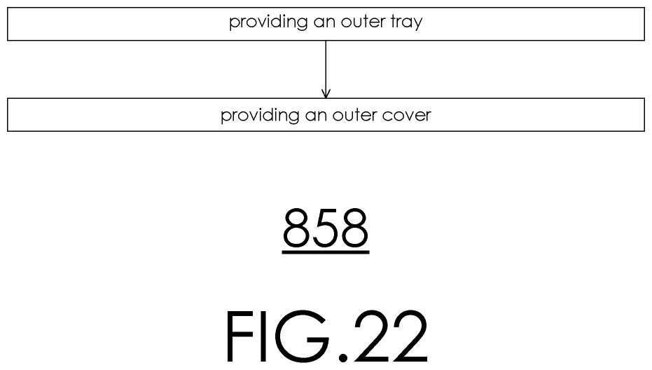

Next, method 800 of FIG. 8 includes an activity 858 of providing an outer container. FIG. 22 illustrates a flow chart for an exemplary embodiment of activity 858 of method 800, according to an embodiment.

Referring to FIG. 22, activity 858 includes a procedure 2271 of providing an outer tray. As an example, the outer tray can be similar or identical to outer tray 201 of FIG. 2.

Activity 858 in FIG. 22 continues with a procedure 2272 of providing an outer cover. As an example, the outer cover can be similar or identical to outer cover 207 of FIG. 2. In some examples, outer cover 207 can be removable couplable to outer tray 201 such that implantation system 200 is sealed such that the sterility of the first container and the implant are maintained until the first container is removed from the outer container.

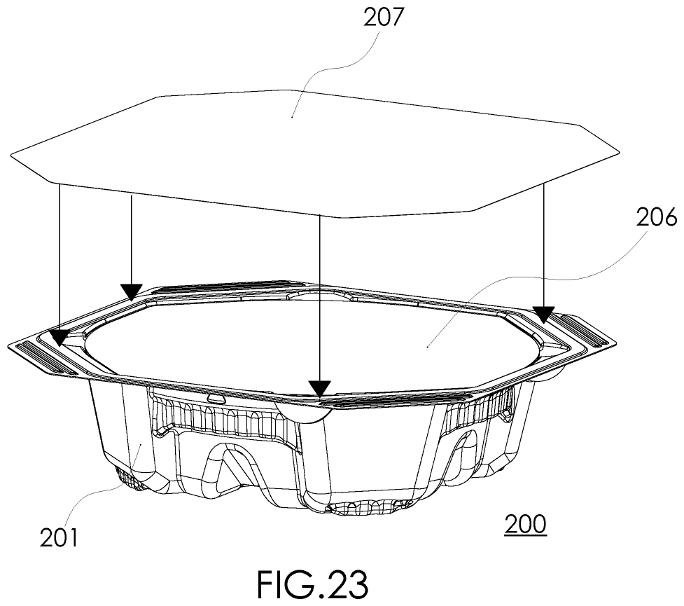

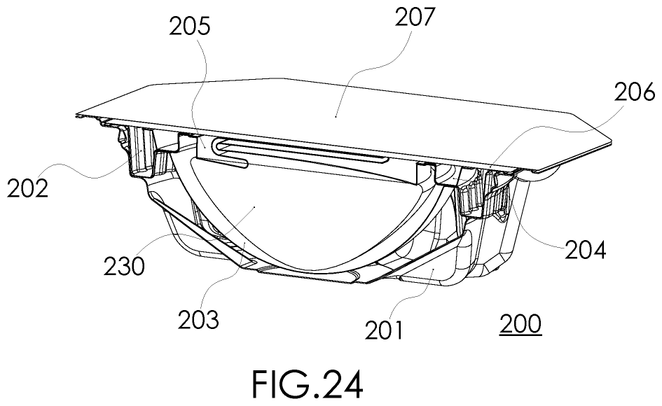

Referring back to FIG. 8, method 800 in FIG. 8 continues with an activity 859 of placing the outer container around the first container. In some embodiments, the outer container is placed around the first container such that the first container is removable from the outer container. In the same or different embodiments, the outer container is sealed and configured such that the sterility of the first container is maintained until the first container is removed from the outer container. FIG. 23 illustrates an isometric view of a breast implantation system 200 before outer cover 207 is coupled to outer tray 201, according to an embodiment. FIG. 24 illustrates a cut-away view of a breast implantation system 200, according to an embodiment.

In one example, outer cover 207 can be surface welded to outer tray 201. In other examples, an adhesive can be used to coupled outer cover 207 to outer tray 201. When outer cover 207 is coupled to outer tray 201, a closed system is created and a sterile environment around the implant first container 210 can be maintained until outer cover 207 is removed in the operating room. Keeping the system closed from the end of the manufacturing process until opened in the operating room will decrease the possibility of bacteria or other foreign materials getting on implant 230, dome 203, and/or funnel 205 before implantation. Using the outer tray and outer cover in addition to the inner cover improves the ease of use of the implantation system compared to embodiments that lack the outer tray and outer cover. That is, if frame 202, dome 203, funnel 205, inner cover 206 are all sterile, first container 210 can be removed from the outer container and placed on a sterile tray or surface in the operating room. Thus, first container 210 would only be touched by medical professionals with their sterile gloved hands and/or other sterile instruments in the operating room. This decreases the risk of infection and increase the ease of use of the implantation system.

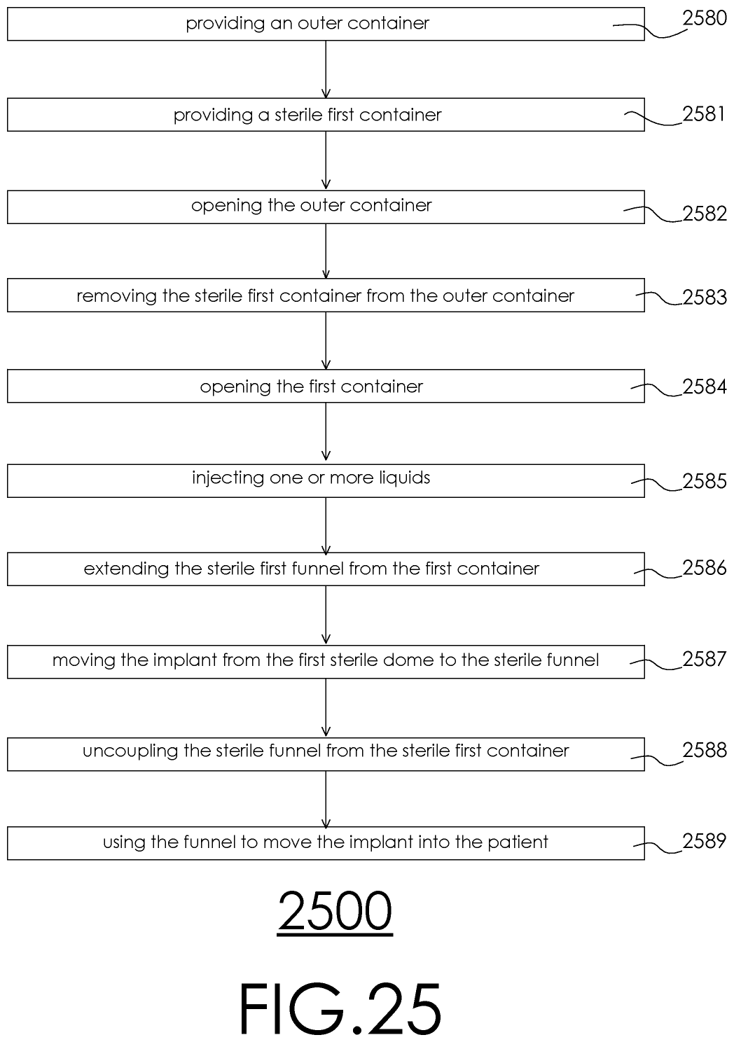

FIG. 25 illustrates a flow chart for an embodiment of a method 2500 of placing a sterile implant in a patient at a point of insertion. Method 2500 is merely exemplary and is not limited to the embodiments presented herein. Method 2500 can be employed in many different embodiments or examples not specifically depicted or described herein. In some embodiments, the activities, the procedures, and/or the processes of method 2500 can be performed in the order presented. In other embodiments, the activities, the procedures, and/or the processes of method 2500 can be performed in any other suitable order. In still other embodiments, one or more of the activities, the procedures, and/or the processes in method 2500 can be combined or skipped.

Referring to FIG. 25, method 2500 includes an activity 2580 of providing an outer container. As an example, the outer container can be similar or identical to the combination of outer tray 201 and outer cover 207 of FIG. 2.

Method 2500 in FIG. 25 continues with an activity 2581 of providing a sterile first container. As an example, the first container can be similar or identical to 101 of FIG. 1 or container 210 of FIG. 2. In some embodiments, the outer container is located around the sterile first container such the sterility of the sterile first container is maintained until the outer container is opened. In some embodiments, the sterile first container can include a sterile first dome with a first side and a second side with the implant inside the first container and a sterile funnel with a first end and a second end. The sterile first dome can be the same or similar to dome 105 or 203 of FIGS. 1 and 2. respectively. The sterile funnel can be the same or similar to funnel 120 or 205 of FIGS. 1 and 2, respectively.

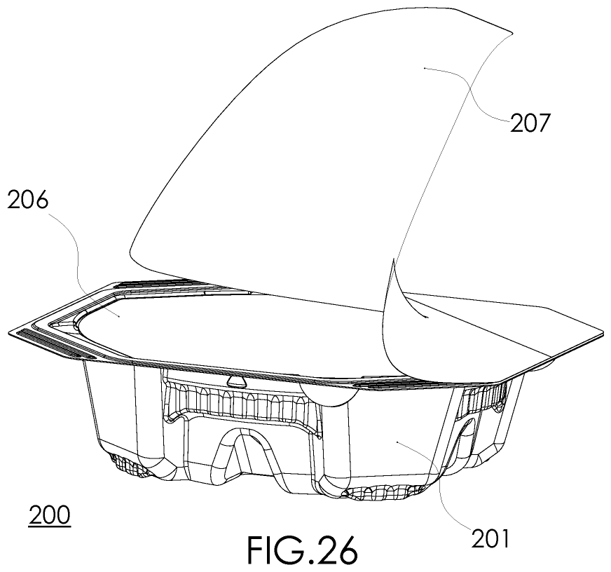

Method 2500 in FIG. 25 continues with an activity 2582 of opening the outer container. FIG. 26 illustrates an isometric view of the opening of outer container 201, according to an embodiment. In some examples, outer cover 207 can be peeled or otherwise removed from outer tray 207 to open the outer container.

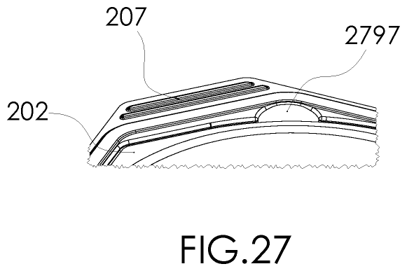

Subsequently, method 2500 of FIG. 25 includes an activity 2583 of removing the sterile first container from the outer container. In some examples, sterile first container 210 can be removed from outer tray 207 after outer cover 207 has been removed by turning outer tray 207 over and dropping first container 210 on a sterile surface in the operating room. In other embodiments, outer tray 207 can include one or more mechanism to allow a medical professional to remove first container 210 from outer tray 207. FIG. 27 illustrates a partial view of frame 202 and outer tray 207 with a finger picker 2797, according to an embodiment. In some examples, outer tray 207 can have two or more finger pickers. In these examples, a medical professional would place her or his fingers in the finger pickers and pick first container 210 out of outer tray 207. In many examples, first container 210 is placed on a sterile surface after removal from outer tray 207 to maintain the sterility of first container 210.

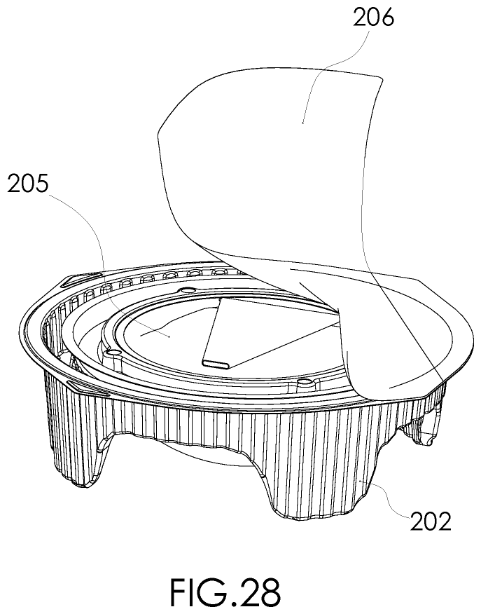

Next, method 2500 of FIG. 25 includes an activity 2584 of opening of the first container. In some examples, opening the first container can include removing an inner cover from the first container. FIG. 28 illustrates an isometric view of the opening of first container 210, according to an embodiment. In some examples, inner cover 206 can be peeled or otherwise removed from frame 202 to open the first container 210.

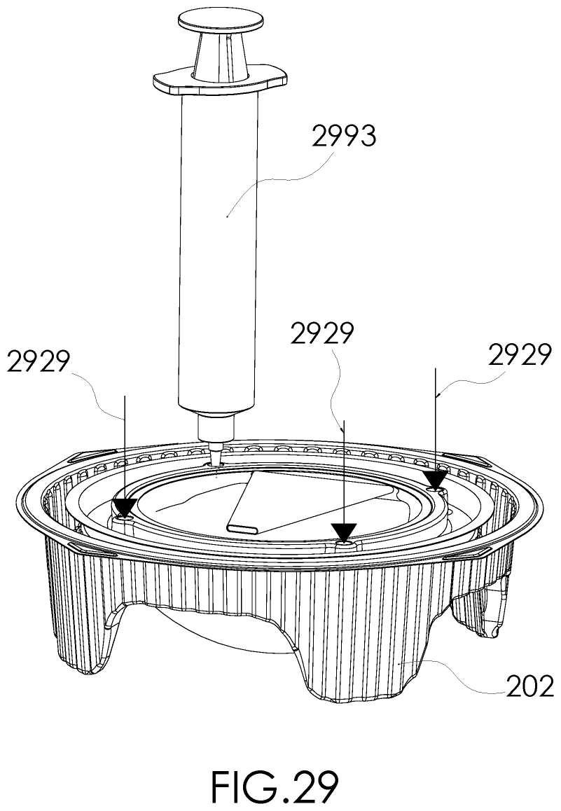

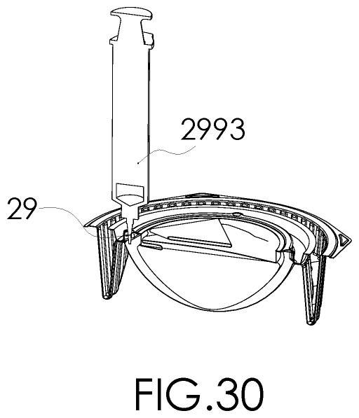

Referring to FIG. 25, method 2500 includes an activity 2585 of injecting one or more liquids. In some examples, frame 202 can include one ore more injection ports 2929 that are located adjacent to flaps 1241 (FIG. 12) on funnel 205. FIG. 29 illustrates an isometric view of the injecting of liquid using a syringe 2993 into dome 203 using one of ports 2929, according to an embodiment. FIG. 30 illustrates a cut-away close up view of the injecting of liquid using a syringe 2993 into dome 203 using one of ports 2929, according to an embodiment.

In some examples, the liquid injected into dome 203 can be an antibacterial and/or antiseptic solution to help kill any bacterial or other microbes that on implant 230. While the first container should be sterile, injecting an antibacterial and/or antiseptic solution is another way to ensure and decease the likelihood of any bacterial issues. In various embodiments, the liquid injected into dome 203 can be Betadine® antiseptic solution. In many embodiments, after the liquid is injected into dome 203, a medical professional can shake, roll, or swirl first container 210 to mix the liquid around implant 230 in dome 203. In other embodiments, the medical professional can push on the bottom of dome 203 with one or more sterile instruments or their finger to help ensure that the liquid gets under implant 230, which is sitting in dome 203.

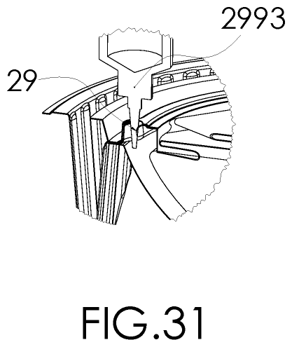

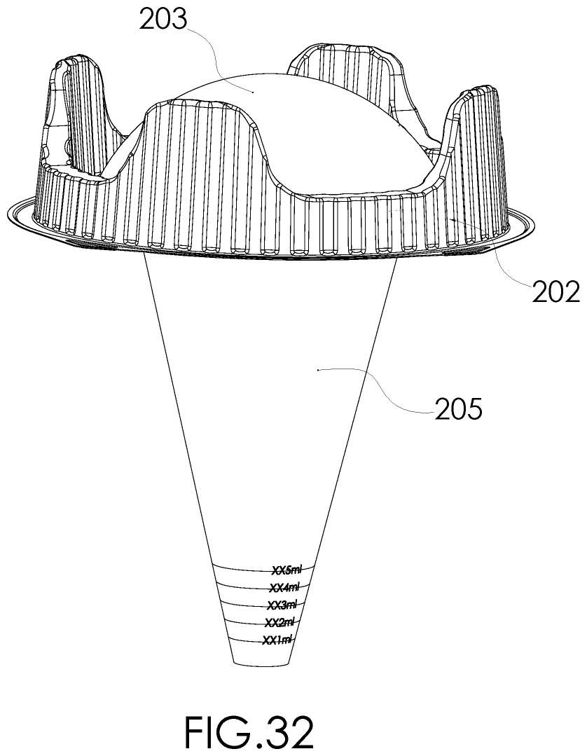

Method 2500 in FIG. 25 continues with an activity 2586 of extending the sterile first funnel from the first container. In some examples, the sterile first funnel is extended from the first container without any human contact with the sterile first funnel. In various embodiments, first container 210 can be rotated one hundred and eighty degrees along the z-axis with dome 203 moving from the bottom to the top and funnel 205 being on the bottom after the rotation is complete. By rotating first container 210, gravity can cause funnel 205 to unfold and extend. FIG. 31 illustrates an isometric view of first container 210 during the rotation process, according to an embodiment. FIG. 32 illustrates an isometric view of first container 210 after rotation, according to an embodiment.

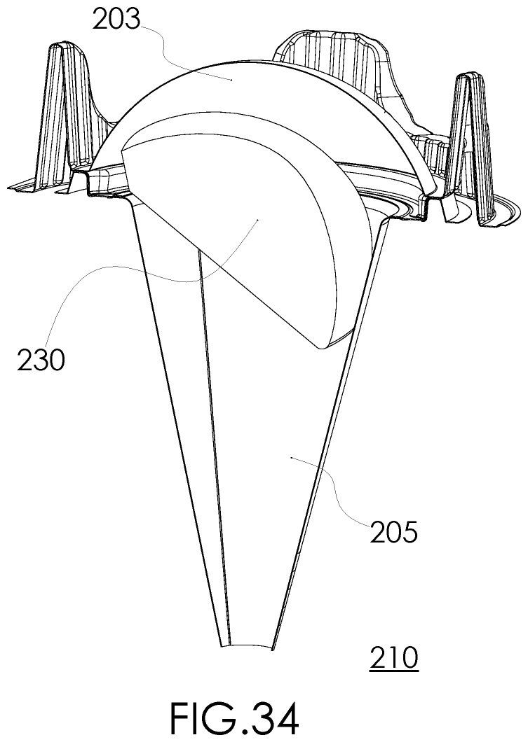

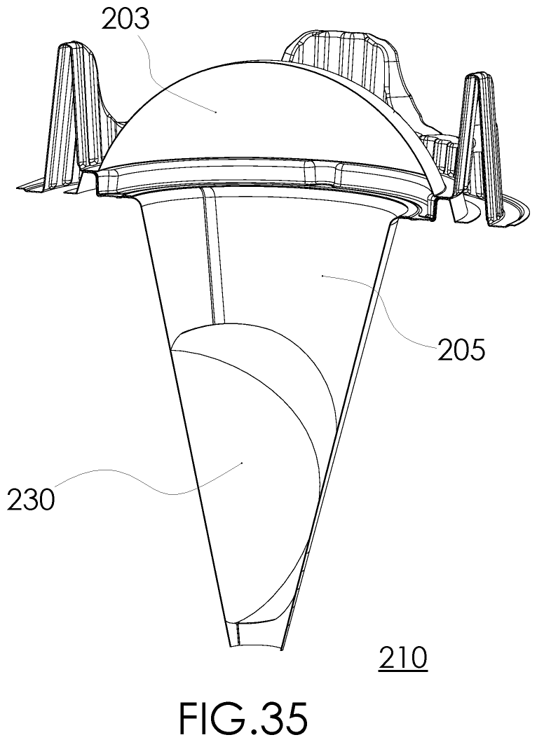

Subsequently, method 2500 of FIG. 25 includes an activity 2587 of moving the implant from the first sterile dome to the sterile funnel. In many examples, implant 230 can move from dome 203 to funnel 205 without any human contact with the implant 230. In some examples, gravity can move implant from dome 203 to end 1246 (FIG. 13) of funnel 205 without any help by any person or use of surgical instruments. In other examples, medical personnel can shake or wiggle first container 210 to begin the descension of implant 230. However, in all of these examples, breast implantation system 200 is still a closed system and there has been no human contact of implant 230 or contact with any surgical instrument since the manufacturing process was completed. This no-touch, closed system is a significant improvement over existing methods because it provides a greater level of protect against bacterial or other contamination of implant 230 before implant 230 enters the patient's body. FIG. 33 illustrates a cut-away view of first container 210 before descension of implant 230 into funnel 205, according to an embodiment. FIG. 34 illustrates a cut-away view of first container 210 during descension of implant 230 into funnel 205, according to an embodiment. FIG. 35 illustrates a cut-away view of first container 210 after descension of implant 230 into funnel 205, according to an embodiment.

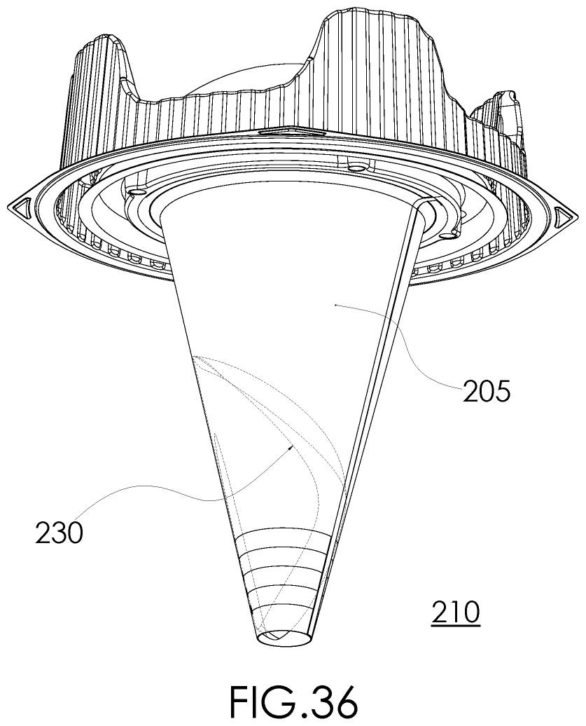

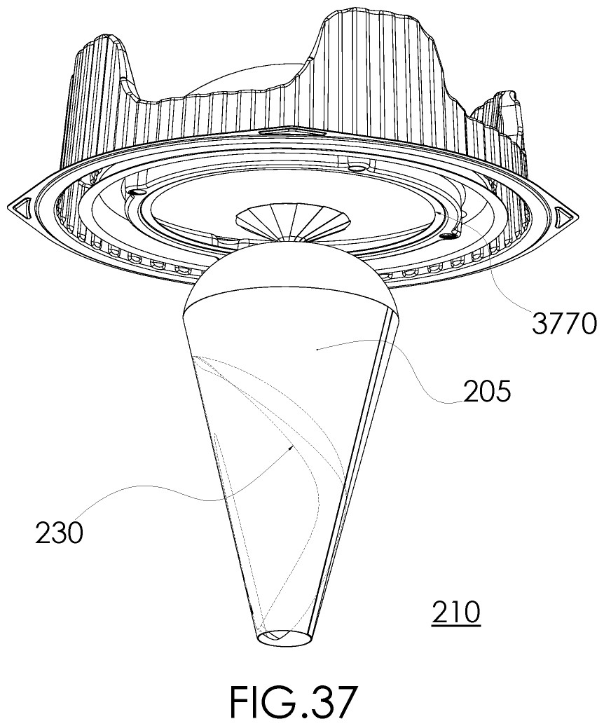

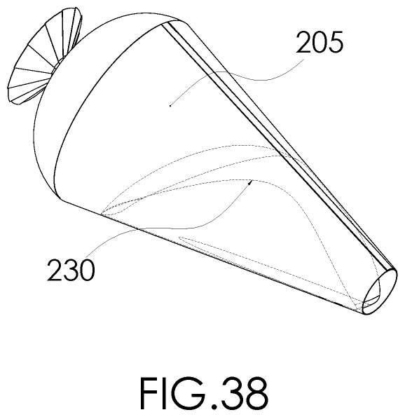

Next, method 2500 of FIG. 25 includes an activity 2588 of uncoupling the sterile funnel from the sterile first container. In some examples, funnel 205 is uncoupled from first container 210 before using funnel 205 to move implant 230 into the patient. In other examples, funnel 205 is not uncoupled from first container 210. FIG. 36 illustrates an isometric view of first container 210 before detachment of funnel 205, according to an embodiment. FIG. 37 illustrates an isometric view of first container 210 during detachment of funnel 205, according to an embodiment. FIG. 38 illustrates an isometric view of funnel 205 after detachment of 205 funnel from first container 210, according to an embodiment.

In some embodiments, funnel 205 can be detached from first container 210 along perforations 1242 (FIG. 12). For example, a surgeon or other medical professional can pinch funnel 205 along perforations 1242 and rotate frame 202 to tear funnel 205 from frame 202 along perforations 1242. In other examples, other mechanisms can be used to detach funnel 205 from first container 210.

Subsequently, method 2500 of FIG. 25 includes an activity 2589 of using the funnel to move the implant into the patient. In many examples, the funnel 203 can be used to move implant 230 into the patient without any human contact (or contact with surgical instruments) to implant 230, other than contact with the patient at the point of insertion. The insertion of implant 230 into the patient can be performed using techniques and processes, well known in the surgical field.

Although the invention has been described with reference to specific embodiments, it will be understood by those skilled in the art that various changes may be made without departing from the spirit or scope of the invention. Accordingly, the disclosure of embodiments of the invention is intended to be illustrative of the scope of the invention and is not intended to be limiting. It is intended that the scope of the invention shall be limited only to the extent required by the appended claims. For example, to one of ordinary skill in the art, it will be readily apparent that activities 850-859 of FIGS. 8, and 2580-2589 of FIG. 25 may be comprised of many different activities, procedures and be performed by many different modules, in many different orders that any element of FIG. 1 or 2 may be modified and that the foregoing discussion of certain of these embodiments does not necessarily represent a complete description of all possible embodiments.

All elements claimed in any particular claim are essential to the embodiment claimed in that particular claim. Consequently, replacement of one or more claimed elements constitutes reconstruction and not repair. Additionally, benefits, other advantages, and solutions to problems have been described with regard to specific embodiments. The benefits, advantages, solutions to problems, and any element or elements that may cause any benefit, advantage, or solution to occur or become more pronounced, however, are not to be construed as critical, required, or essential features or elements of any or all of the claims, unless such benefits, advantages, solutions, or elements are stated in such claim.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

What is claimed is:

1. An implantation system for placing an implant into a patient during a surgical procedure wherein the implant is sterile, the implantation system comprising:
a first container with a first side and a second side and configured to hold the implant inside the first container; and
a funnel with a first end and a second end;
wherein:
an inner surface of the first container and an inner surface of the funnel are sterile;
the first end of the funnel is coupled to the second side of the first container such that the implant can move inside the funnel from the first container and the funnel is further configured such that the implant can be removed from the funnel at the second end of the funnel; and
the first container and the funnel are configured such that the sterility of the implant is maintained until the implant is removed the second end of the funnel.

2. The implantation system of claim 1, wherein the first container is sterile and further comprising:
an outer container located around the first container such that the first container is removable from the outer container and the outer container is sealed and configured such that the sterility of the first container is maintained until the first container is removed from the outer container.

3. The implantation system of claim 2, wherein
the outer container comprises:
an outer tray; and
an outer cover that is removable coupled to the outer tray such that sealed such that the sterility of the first container and the implant are maintained until the first container is removed from the outer container.

4. The implantation system of claim 1, wherein:
the funnel is sterile;
the first container comprises:
a frame with an inner surface and coupled to the funnel and such that the inner surface of the frame is sterile;
a dome with an inner surface coupled to the frame such that the implant rests in the dome and the inner surface of dome is sterile; and
an inner cover that is configured to removably couple to the frame such sterility of the funnel is maintained until such inner cover is removed from the frame; and
the inner surface of the first container comprises the inner surface of the frame and the inner surface of the dome.

5. The implantation system of claim 1, wherein:
the funnel contains is coupled to the container at a first side;
the funnel includes an opening at a second side; and
a size of the opening is substantially the same size as the volume of the implant.

6. The implantation system of claim 1, wherein:
the funnel is folded; and
the first container and the funnel are configured such that the funnel can be unfolded, and the implant can move from the first container into the funnel without the funnel being touched by a person or any surgical instrument.

7. The implantation system of claim 1, wherein:
the funnel is removable from the first container.

8. The implantation system of claim 1, wherein:
the first container comprises one or more unsealable flaps to allow injection of one or more liquids into the first container while maintaining the sterility of the implant and the inner surface of the container.

9. The implantation system of claim 8, wherein:
the one or more unsealable flaps are configured such that the one or more unsealable flaps are openable without detaching any parts of the first container.

10. The implantation system of claim 1, further comprising the implant and wherein the implant is a breast implant.

11. A method of producing an implantation system for an implant, comprising:
sterilizing the implant;
providing a first container with a first side and a second side;
providing a funnel with a first end and a second end where the funnel is further configured such that the implant can be removed from the funnel at the second end of the funnel;
sterilizing at least an inner surface of the first container and an inner surface of the funnel;
placing the implant into the first container; and
coupling the first container to the funnel such that the first end of the funnel is coupled to the second side of the first container such that the implant can move inside the funnel from the first container and that the sterility of the implant is maintained until the implant is removed the second end of the funnel.

12. The method of claim 11, further comprising:
providing an outer container; and
placing the outer container around the first container and the funnel such that the first container and funnel are removable from the outer container and the outer container is sealed and configured such that the sterility of the first container and funnel are maintained until the first container and funnel are removed from the outer container,
wherein:
sterilizing at least an inner surface of the first container and an inner surface of the funnel comprises sterilizing an outer surface and the inner surface of the first container and the funnel.

13. The method of claim 12, wherein:
providing the outer container comprises:
providing an outer tray; and
providing an outer cover that is removable coupled to the outer tray such that the sterility of the first container and the funnel are maintained until the first container and funnel are removed from the outer container.

14. The implantation system of claim 11, wherein:
providing the first container comprises:
providing a frame with an inner surface and removably coupled to the funnel;
providing a dome with an inner surface coupling to the dome to the frame such that the implant rests in the dome;
providing an inner cover; and
removably coupling to the frame to the inner cover such that sterility of the funnel is maintained until such inner cover is removed from the frame; and
sterilizing at least the inner surface of the first container and an inner surface of the funnel comprises: sterilizing the inner surface the first container and the inner surface of the funnel; and
the inner surface of the first container comprises the inner surface of the frame and the inner surface of the dome.

15. A method of placing a sterile implant in a patient at a point of insertion, the method comprising:
unsealing a sterile first container to expose a sterile funnel wherein sterile first container comprises:
a sterile funnel with a first end and a second end;
a sterile dome with a first side and a second side with the sterile implant inside the sterile dome;
moving the sterile implant from the sterile dome to the sterile funnel without any human contact with the implant; and
using the sterile funnel to move the sterile implant into the patient without any human contact to the sterile implant other than contact with the patient at the point of insertion.

16. The method of claim 15, further comprising:
opening an outer container wherein the outer container located around the sterile first container such the sterility of the sterile first container is maintained until the outer container is opened; and
removing the sterile first container from the outer container.

17. The method of claim 15, further comprising:
uncoupling the sterile funnel from the sterile first container before using the sterile funnel to move the implant into the patient.

18. The method of claim 15, further comprising:
injecting one or more liquids into the sterile dome using one or more injection ports in the sterile first container.

19. The method of claim 18 wherein:
the one or more liquids comprise betadine.

20. The method of claim 15, further comprising:
removing an inner cover from the sterile first container;
extending the sterile funnel from the sterile first container without any human contact with the sterile funnel.
